(19) United States
(12) Patent Application Publication
(10) Pub. No.: US 2024/0099658 A1
(43) Pub. Date: Mar. 28, 2024

(54) ARTICLE OF CLOTHING FOR SUPPORTING WEARABLE MEDICAL DEVICE

(71) Applicant: WEST AFFUM HOLDINGS DAC, DUBLIN (IE)

(72) Inventors: Pamela F. Breske, Newcastle, WA (US); Alexis Cummings, Kirkland, WA (US); Kimberly L. Malone, Monroe, MI (US); Christina K. House, Lee's Summit, MO (US); Jennifer D. Imerini, St. Augustine, FL (US); Traci S. Umberger, Kirkland, WA (US); Brian D. Webster, Mercer Island, WA (US); Krystyna Szul, Seattle, WA (US)

(73) Assignee: WEST AFFUM HOLDINGS DAC, DUBLIN (IE)

(21) Appl. No.: 18/474,427

(22) Filed: Sep. 26, 2023

Related U.S. Application Data
(60) Provisional application No. 63/410,208, filed on Sep. 26, 2022.

(51) Int. Cl. A61B 5/00; A61B 5/256; A61B 5/282

(52) U.S. Cl. CPC A61B 5/6805; A61B 5/256; A61B 5/282

(57) ABSTRACT

The disclosure is directed at an accessory for a garment that may be worn in conjunction with a wearable medical device (WMD). Certain embodiments of the accessory enable customization of the garment for disparate physical anatomies of various patients. Other embodiments of the accessory enable customization of the garment for aesthetics and comfort of various patients. Non-exhaustive examples of such a WMD include wearable cardioverter defibrillators and wearable monitoring devices.

### CROSS-REFERENCE TO RELATED APPLICATION

The present application claims priority to and the benefit of U.S. Provisional Application No. 63/410,208, filed Sep. 26, 2022, and entitled Article of Clothing for Supporting Wearable Medical Device, the entire disclosure of which is hereby incorporated by reference herein in its entirety for all purposes.

### SUMMARY OF THE DISCLOSURE WITH BACKGROUND

Sudden Cardiac Arrest (SCA) accounts for more than 300,000 deaths in the United States alone, annually. Some of the high-risk population, such as post-myocardial infarction patients, who have implantable cardioverter defibrillators (ICD) have been shown to have a reduced mortality rate from those in the high-risk population without ICDs. However, there are patients who are at high risk but who are, for a variety of reasons, deemed inappropriate candidates for ICDs, but who may still benefit from automatic external defibrillation. A wearable cardioverter defibrillator (WCD) is a device designed for patients at high risk of SCA but who are not immediate candidates for an ICD. The WCD is a medical device that is worn externally and includes a fabric garment assembly fitted to a patient's chest which holds electrodes in place. The WCD is capable of automatic detection and defibrillation of ventricular tachycardia (VT) or ventricular fibrillation (VF).

However, WCDs have been designed as unisex devices that may not be comfortable for a female patient's anatomy. WCDs generally require a female patient to either wear a bra over the WCD, which may result in discomfort, or forgo a bra, which may lead to lack of support for the female patient's breasts. Other types of external wearable medical devices (WMDs), such as wearable medical monitoring devices and the like, also suffer from several of the same problems as WCDs.

None of the subject matter discussed in this section is necessarily prior art and may not be presumed to be prior art simply because it is presented in this section. Any reference to any prior art in this description is not, and should not be taken as, an acknowledgment or any form of suggestion that such prior art forms parts of the common general knowledge in any art in any country. Along these lines, any recognition of problems in the prior art discussed in this section or associated with such subject matter should not be treated as prior art, unless expressly stated to be prior art. Rather, the discussion of any subject matter in this section should be treated as part of the approach taken towards solving the particular problems identified. This approach in and of itself may also be inventive.

### BRIEF DESCRIPTION OF THE DRAWINGS

FIG. 1 is a conceptual diagram of a patient wearing an exemplary accessory to a Wearable Medical Device (“WMD”) garment positioned over the garment, made according to embodiments;

FIG. 2 is another embodiment of an accessory to a WMD with a back cushion extender, made in accordance with this disclosure;

FIG. 3 is an alternative embodiment of the accessory shown in FIG. 2, made in accordance with this disclosure;

FIG. 4 is another embodiment of an accessory to a WMD tailored to patients having different anatomies, made in accordance with this disclosure;

FIG. 5 is yet another embodiment of an accessory to a WMD that includes flaps for access to the patient's breast area, made in accordance with this disclosure;

FIG. 6 is an alternative embodiment of the accessory shown in FIG. 5 adapted to accept various accessories based on the patient's particular anatomy, made in accordance with this disclosure;

FIG. 7 is still another embodiment of an accessory to a WMD that includes lacing in the upper portion, made in accordance with this disclosure;

FIG. 8 is an alternative embodiment of the accessory shown in FIG. 7, made in accordance with this disclosure;

FIGS. 9A, 9B, and 9C are illustrations of still another embodiment of an accessory for a WMD adapted for greater comfort for the patient, made in accordance with this disclosure;

FIGS. 10A and 10B are illustrations of an alternative embodiment of the accessory shown in FIGS. 9A-C, made in accordance with this disclosure;

FIG. 11 is an alternative embodiment of the accessory shown in FIGS. 10A-B, made in accordance with this disclosure;

FIGS. 12A and 12B are illustrations of still another embodiment of an accessory to a WMD that includes enhanced breast support, made in accordance with this disclosure;

FIGS. 13A and 13B are illustrations of an alternative embodiment of the accessory shown in FIGS. 12A-B, made in accordance with this disclosure;

FIG. 14 is an illustration of still another embodiment of an accessory for a WMD customized for the patient, made in accordance with this disclosure;

FIG. 15 is an alternative embodiment of the accessory shown in FIG. 14, made in accordance with this disclosure;

FIG. 16 is an illustration of still another embodiment of an accessory for a WMD incorporating various fasteners, made in accordance with this disclosure; and

FIG. 17 is an alternative embodiment of the accessory shown in FIG. 16, made in accordance with this disclosure.

FIG. 18 is an illustrative WMD in which embodiments of the disclosure may be implemented.

FIG. 19 is another illustrative WMD, showing various monitoring components, in which embodiments of the disclosure may be implemented.

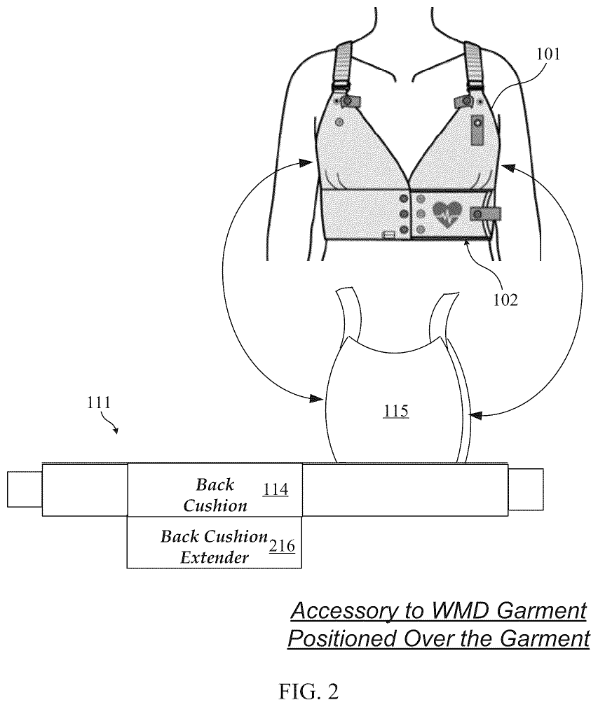

Some figures may have alternative views depicted as “A”, “B”, “C”, etc. In such a case, the alternative views may be referred to collectively by reference to the figure number only. For example, if FIG. 2 includes alternative views 2A and 2B, those alternative views may be each referred to individually as FIG. 2A and FIG. 2B, and collectively as FIG. 2.

### DETAILED DESCRIPTION

This disclosure is directed at an accessory for a garment that may be worn in conjunction with a wearable medical device (WMD). The accessory enables customization of the garment for disparate physical anatomies of various patients. Non-exhaustive examples of such a WMD include wearable cardioverter defibrillators and wearable monitoring devices.

An external WMD is typically worn by a patient for an extended period of time. To facilitate wearing the WMD, a garment for securing the WMD (or components of the WMD) to the patient is provided. Such a garment may be implemented as any article of clothing, such as a vest, a belt, a wrap, a bra, a halter, etc. To ensure a patient's comfort and care needs while also adequately monitoring the patient's vital statistics, such garment may include attachments, pockets, hooks, snaps, zippers, hook-and-loop fasteners, etc.

One goal of the garment is that it can adequately secure sensors for the WMD in place and in contact with the body, over an extended wear time, for monitoring cardiac signals. To achieve that goal, the garment should be comfortable and, when appropriate, address additional care needs a patient may have. Various patients who are prescribed a WMD may have differing needs. For example, in some cases, a WMD may be prescribed for patients who previously suffered from a cardiac event. In some cases, the patient could be a breastfeeding mother. In some cases, a WMD may be prescribed for patients who have had chest or breast augmentation. In some cases, a WMD may be prescribed for patients suffering from another disease, such as cancer.

The garment may also be customizable to the patient by adding an accessory. The accessory may be designed to be added to or included in a garment for a WMD. The accessory may provide additional customization or features to the garment. For example, cancer patients may have medication ports for chemotherapy installed in their upper chest area and may need easy access to those ports as well as proper care and infection prevention. Patients who have undergone a mastectomy may benefit from a garment that accommodates a breast prosthesis, facilitates fluid drainage, and/or provides compression. Patients who have undergone breast augmentation may need additional support for the breast tissue and/or to prevent it from interfering with cardiac sensors.

Providing patients with a garment that can support their medical monitoring needs as well as other care and comfort needs without sacrificing aesthetics and comfort is therefore highly desirable. A garment for a WMD may be constructed to aid the healing, ease of use, peace of mind, and overall patient satisfaction. One example of a garment providing support for a WCD can be found in U.S. Pat. No. 10,926,080, issued Feb. 23, 2021 and entitled “Wearable Cardioverter Defibrillator with Breast Support,” which is incorporated by reference herein for all purposes. In some embodiments, such a garment can include modular parts, modifiable and/or accessorizable, per the patient's needs, comfort, and desires.

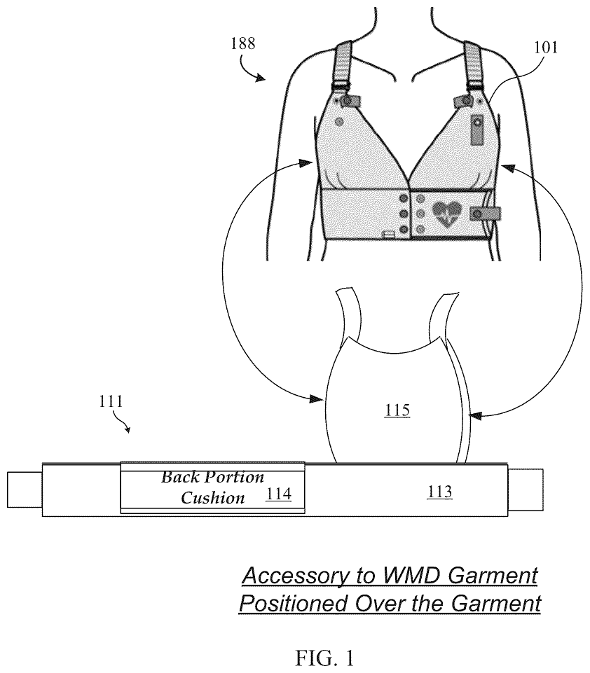

FIGS. 1-4 illustrate various embodiments of an accessory for a WMD garment to provide additional coverage. Beginning with FIG. 1, a WMD garment 101 is shown as worn by a patient 188. One embodiment of an accessory 111 includes a belt portion 113, which can be secured over the garment 101 and house one or more ECG electrodes (not shown). A back portion 114 of the accessory 111 can include a cushioning material for added patient comfort. The accessory 111 can be adjusted and tightened around the garment 101. In that manner, the accessory 111 can help prevent the ECG electrodes from losing contact with the skin. The WMD's accessory upper portion 115 extends from the belt portion 113 and includes sides that are attachable to the garment 101. In some embodiments, the upper portion 115 can be repositioned along the belt portion 113 to allow a desired alignment with the upper body that the patient wishes to cover and/or support. The positioning of the upper portion 115 along the belt portion 113 and/or sides of the garment can be achieved by any one or more of several mechanisms, such as clips, clasps, a zipper, lacing, hook-and-loop fasteners, hooks, etc. In some embodiments, the upper portion 115 of the accessory 111 can also be secured in the back with additional straps extending from the sides of the upper portion 115.

In various embodiments, the upper portion 115 of the accessory 111 may be configured to receive padding, breast prosthesis, fluid drainage absorbent, compression pad material, tubing to access a chemo port or drain fluids, and the like.

As shown in FIG. 2, other embodiments of the accessory 111 may include a back cushion extender 216. In such embodiments, the back cushion extender 216 may be configured to extend from the back portion cushion 114 and fold underneath a belt portion 102 of the garment 101, and may be folded in between the patient's skin and the inner portion (skin facing layer) of the garment belt 102. In such embodiments, the garment belt 102 may be configured to house electronics, and/or cables adjacent to the patient's back. In some cases, the patient may find additional comfort from the cushion extender 216, which can fold underneath the garment belt 102 where an electronics box and/or cable may cause discomfort. In various embodiments, the cushion extender 216 may be secured in place via snaps, clips, hook-and-loop fasteners, etc., so that it does not interfere with sensing or defibrillating electrodes.

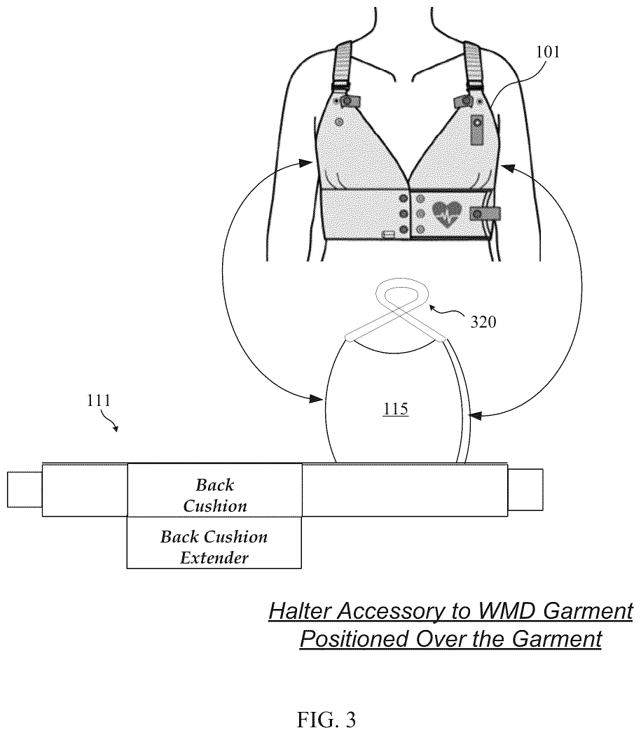

As shown in FIG. 3, the upper portion 115 of the accessory 111 may be configured to secure to the patient's chest in many different ways. In various embodiments, the accessory 111 may be attachable to the WMD garment 101 and the accessory 111 may have one or more strap configurations. These may be implemented using alternative/replaceable straps 320 having different designs, colors, patterns, and configurations. The straps 320 may be replaceable according to the patient's preferences for style, aesthetics, function, comfort, etc. For example, the straps 320 may be shoulder straps, cross-straps, halter-like strap, or the like.

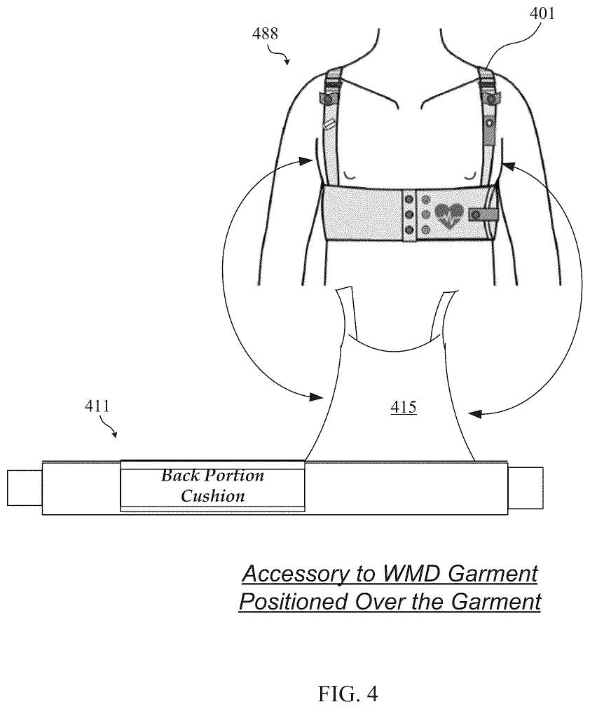

As shown in FIG. 4, the WMD garment accessory 411 may be tailored to patients having different anatomies. For instance, the accessory 411 may be tailored to patients by gender, such as by implementing the upper portion 415 as tank top-like for female patients or, alternatively, vest-like for male patients. In addition, the accessory 411 may be implemented to mimic and be customized to a more business or casual attire. As shown in FIG. 4, the accessory 411 may include a vest-like upper portion 415 for wear by a male patient 488 having a garment 401 that is configured without breast support features.

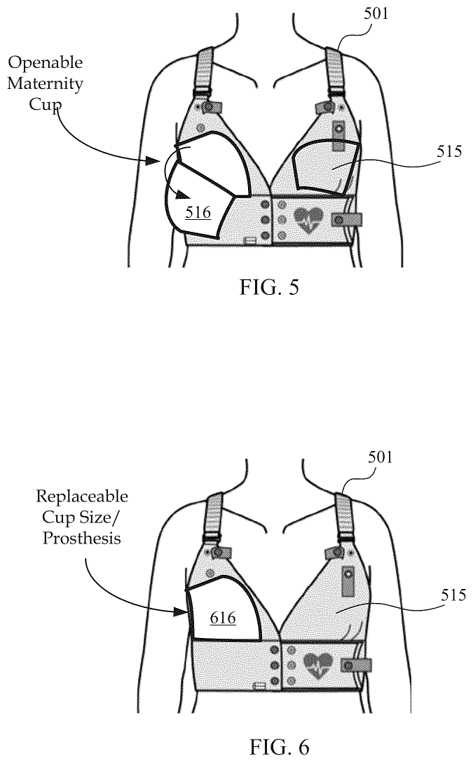

In alternative embodiments, the garment 501 itself may be configured to directly receive accessories in accordance with the teachings of this disclosure. For instance, as shown in FIG. 5, an upper portion 515 of the garment 501 may be configured with a flap 516 that may open for access to the patient's breast, such as for breastfeeding and the like. In addition, as shown in FIG. 6, the upper portion 515 of the garment 501, may be configured to receive various accessories 616 based on the patient's specific anatomy or desires, such as padding, a breast prosthesis, fluid drainage absorbent, compression pad material, or tubing to access a chemo port or drain fluids. These are but a few of the many accessories which may be incorporated into the garment 501 to customize it for the needs, desires, and comfort of various patients.

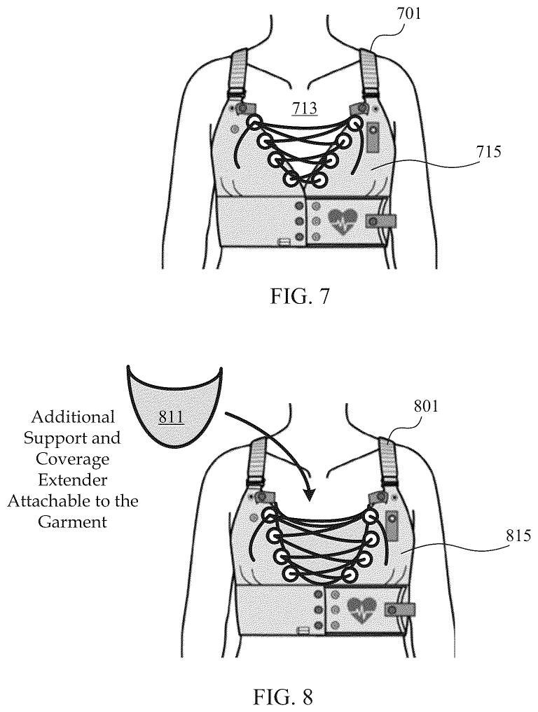

For various reasons, such as modesty, some patients may desire additional coverage or comfort from a WMD garment. In such cases, accessories may be implemented for use in connection with a WMD garment that are tailored specifically to various needs or desires. For example, as shown in FIG. 7, a WMD garment 701 may be implemented with an adjustable lacing accessory 713 that may be used to cinch the upper portion 715 of the garment 701. In another example, as shown in FIG. 8, a modesty panel 811 (e.g., a chemisette) may be implemented that may be inserted at the front of the WMD garment 801 and attachable to the rim of the breast cups 815 to cover the middle chest of the patient. In addition, as shown in FIG. 8, multiple different accessories may, but need not, be used together to further tailor the garment 801 specifically to the patient's particular situation.

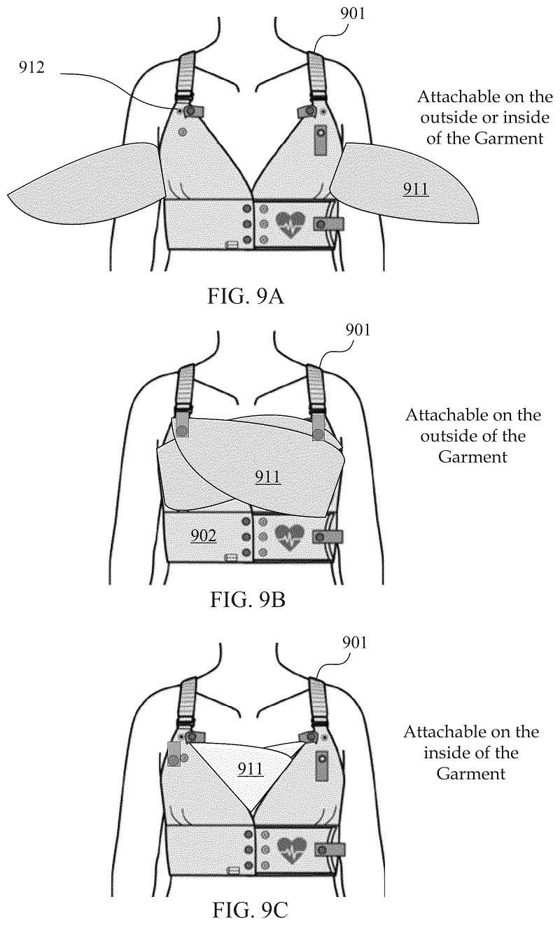

Turning now to FIGS. 9A-C, alternative embodiments of the accessory may be implemented to provide additional coverage and comfort to various patients. For example, as shown in FIGS. 9A-C, accessory flaps may be provided to enable compressing the breast tissue, such as for comfort, or to enable moving the breast tissue away from WMD sensors. In many instances, the WMD sensors are highly sensitive to movement, which introduces noise and interference in the sensor signal. By providing additional support, the accessory flaps may reduce noise and sensor interference.

As shown in FIG. 9A, a female patient is wearing a WMD garment 901 with a wrap accessory 911. In this example, the wrap accessory 911 may attach to the side and/or the back of the garment 901. As illustrated in FIG. 9A, the wrap accessory 911 is attached on the outside of the garment 901. In addition, one end of the wrap accessory 911 is attached at the side or back of the garment 901. The other end of the wrap accessory 911 is wrapped across the front of the patient and attached at an attachment point 912 (e.g., a shoulder strap of the garment 901). In this manner, and as shown in FIG. 9B, the wrap accessory 911 is used to lift the breast tissue up and attach to the attachment point 912 (e.g., the shoulder strap or underarm portion of the garment 901) at the other end. In this way, the breast tissue may be secured up and away from the belt 902 of the garment 901, which may include monitoring sensors (e.g., ECG sensors).

As shown in FIG. 9C, the wrap accessory 911 may alternatively be attached to the inside of the WMD garment 901. Wrap accessories implemented in this manner may provide additional alternatives to further enhance comfort and/or performance.

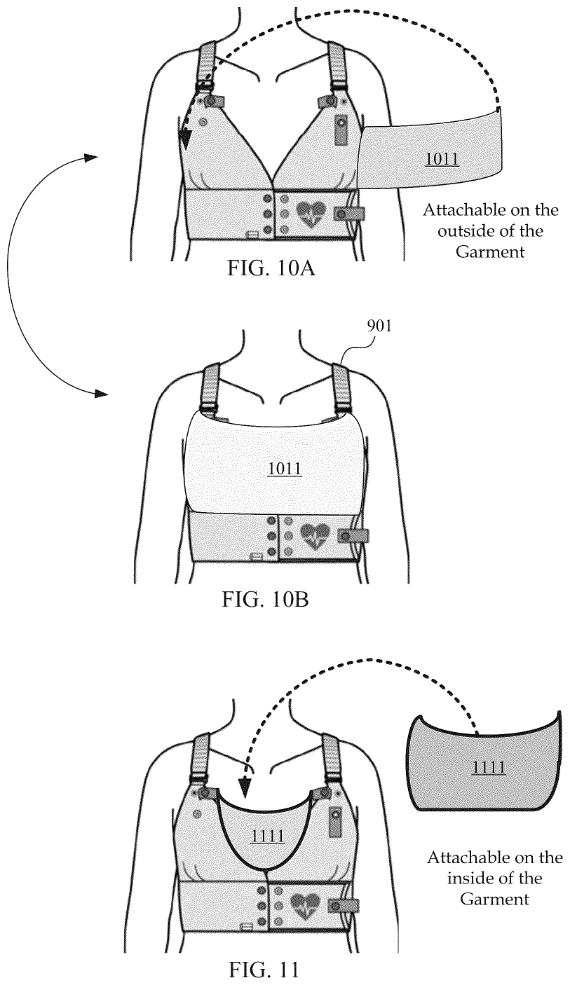

Turning now to FIGS. 10A-B, another alternative wrap accessory 1011 may be implemented as a one-piece wrap in contrast to the two-piece wrap illustrated in FIGS. 9A-C. In this embodiment, the wrap accessory 1011 may attach to the garment 901 at either side (or back) as shown in FIG. 10A, wrap across the front of the patient, and attach at the other side (or back) of the garment 901 as shown in FIG. 10B. Such one-piece wrap accessories help to provide front coverage while also lifting the breast tissue away from the WMD garment 901 belt area, and provide additional options for achieving comfort and enhancing performance of the WMD garment 901.

FIG. 11 illustrates yet another alternative wrap accessory 1111. The embodiment shown FIG. 11 is another one-piece wrap accessory 1111 that attaches inside the upper portion of the garment 901 in a manner similar to the embodiment shown in FIG. 8. The one-piece wrap accessory 1111 may also attach at the sides of the inside of the garment 901 to enable enhanced compression of the breast tissue.

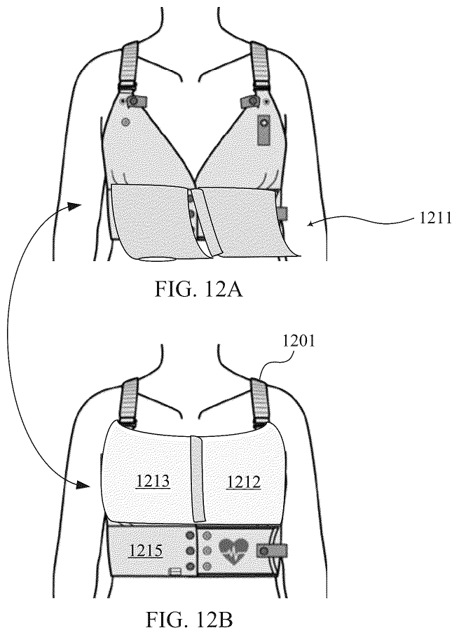

FIG. 12 illustrates yet another alternative accessory 1211 for a WMD garment 1201 with a comfort accessory. As shown in FIGS. 12A and 12B, the garment 1201 includes breast support portions, but in some instances those breast support portions may not provide sufficient support for the patient's comfort. In such a case, the accessory 1211 may be added to the garment 1201 to provide additional support for the patient's breast tissue. In the embodiment illustrated in FIG. 12, the accessory 1211 is implemented as separate portions, a left portion 1212 and a right portion 1213, with each portion covering its respective breast area. However, unlike the embodiments shown in FIGS. 9 and 10, which wrap the patient from the side, the embodiment shown in FIG. 12 attaches to the belt 1215 of the garment 1201 and lifts from below the breasts. As shown in FIG. 12A, each side of the accessory 1211 attaches at the bottom to the belt 1215 of the garment 1201 and may be lifted from the bottom to attach at the top to an attachment point near the shoulder portion of the garment 1201. Lifting from below the breast tissue at the belt 1215 of the garment 1201 enables additional lifting support to be applied to the breast tissue of the patient. In this way, the accessory 1211 helps lift the breast tissue away from the garment belt 1215 which may be housing ECG electrodes or other monitoring sensors.

In addition to the additional lifting support provided by the embodiment illustrated in FIG. 12, the two-part construction of the accessory 1211 provides additional advantages, such as it can compensate for uneven breast tissue, prosthesis, chemo port, breastfeeding needs, and the like. Each of the separate portions (i.e., left portion 1212 and right portion 1213) of the accessory 1211 may overlap so that they may be joined and secured in the middle section thereby providing even more chest coverage if desired.

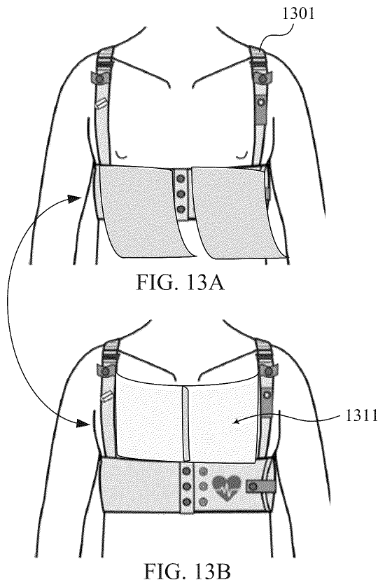

FIG. 13 illustrates an embodiment similar to that shown in FIG. 12. As illustrated in FIG. 13, the accessory 1311 may attach to the belt of a garment 1301 that is without breast support portions. In this way, the accessory 1301 may be used to add breast support to a garment 1301 that does not have breast support as originally manufactured. Such an embodiment provides the advantage that the accessory 1301 and garment 1301 may be tailored to be more or less masculine, feminine, or gender neutral.

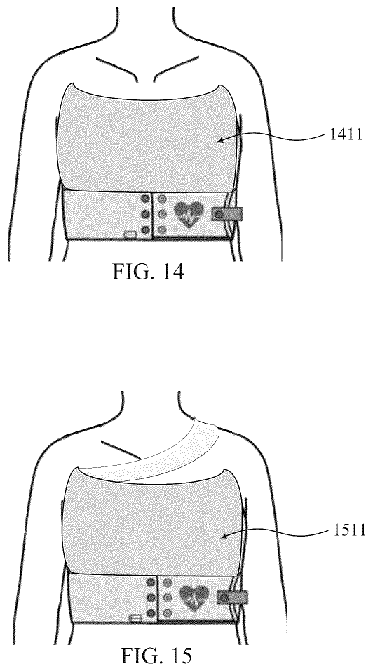

In accordance with the disclosure, embodiments of the accessory may be customized in a variety of ways, such as with various types of straps. For example, as illustrated in FIG. 14 and FIG. 15, different shoulder straps, fasteners, and other accessorizing articles of clothing may be selected to match the patient's outfit or mood. In the example illustrated in FIG. 14, the accessory 1411 may be used in a halter-top style without straps over the shoulder at all, while relying on the support of the underlying garment alone. Alternatively, as illustrated in FIG. 15, the accessory 1511 may be using with a cross-shoulder style strap 1513 extending from one side of the accessory 1511 in the front to the opposite side of the accessory 1511 in the back. In various implementations, shoulder straps and fasteners may be made removable and customizable for different colors and looks. In some embodiments, one or both shoulder straps could be removed altogether, or a single strap could be designed for wear comfort, support, and/or aesthetics.

In some embodiments, different types of fasteners and adjustment mechanisms may be used. For example, FIG. 16 illustrates an embodiment of an accessory 1611 that employs shoulder straps 1612 with a strap fastener 1613. In this embodiment, the strap fastener 1613 includes a clip or clasp structure at one end or both ends that can be fastened at various positions, indicated in FIG. 16 by horizontal ridge structures on the shoulder strap 1612, to provide strap length adjustment. The other end of the strap in this embodiment has a hook-and-loop fastener that provides additional adjustability of the strap length.

In other embodiments of FIG. 16, the strap fastener may incorporate buttons, clips, clasps, hook-and-loop fasteners, or any combination of these fasteners. Such an embodiment provides the additional advantage that patients who may have mobility, dexterity, and/or arthritic disabilities can adjust and tailor the fitment of the garment 1601.

The embodiment shown in FIG. 16 may be adjusted at both ends of the strap fastener 1613 to alter the length of the shoulder strap. However, in alternative embodiments one end of the strap fastener 1613 may be fixedly attached to the garment 1601 while the other end of the strap fastener 1613 is adjustably attached to the shoulder strap. Again, such a fastener may be hook-and-loop fasteners, clasps, clips, buttons, etc.

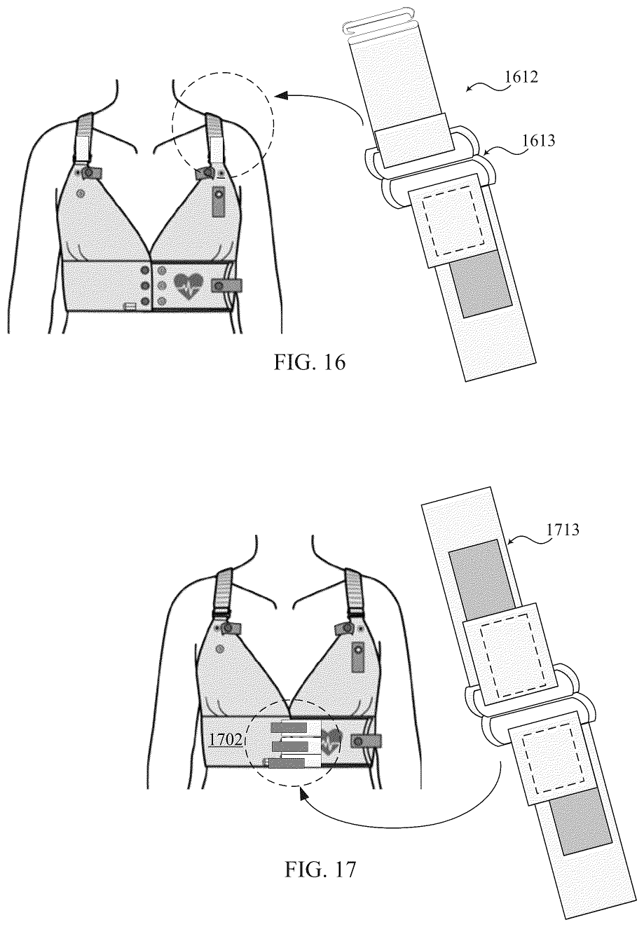

FIG. 17 illustrates an alternative embodiment of a fastener system (torso fastener 1713) for the torso band 1702 (belt) of the WMD garment 1701. As shown in FIG. 17, the torso fastener 1713 is somewhat similar to the strap fastener 1613 illustrated in FIG. 16. However, the torso fastener 1713 is used to provide additional adjustability to the length of the garment torso band 1702. The torso fastener 1713 may employ various types of fasteners, such as hook-and-loop fasteners, on one or both ends of the fastener 1713. In the example illustrated in FIG. 17, three hook-and-loop fasteners are used, similar to the three rows of snap fasteners illustrated in other embodiments. In other embodiments, hook-and-loop fasteners can be used in addition to buttons, snaps, hooks, zippers, and the like to finetune fitment of the torso band 1702 against the patient's skin. In addition, the torso fastener 1713 shown in FIG. 17 may be used to provide additional length for the torso band 1702 for patients who may have more girth in the midsection than other patients.

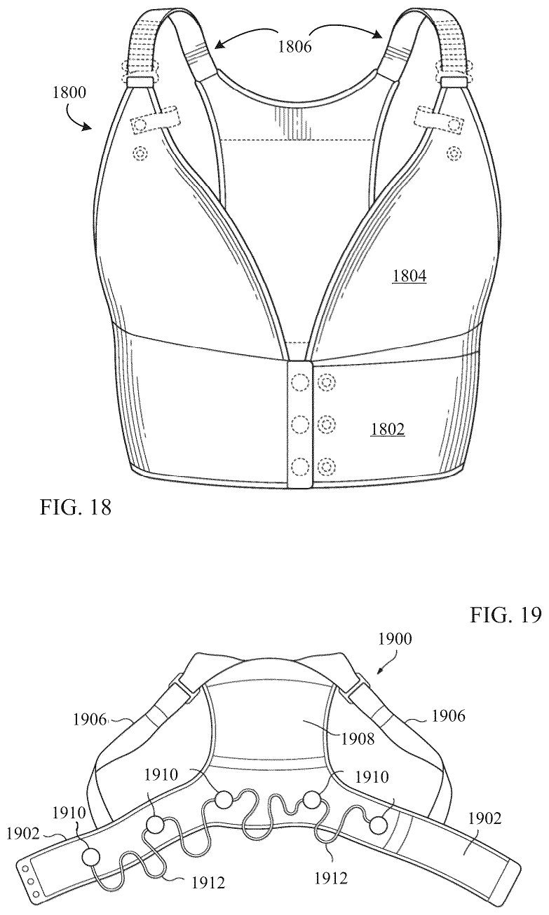

The various embodiments disclosed above may be adapted for use with various Wearable Medical Devices. However, in one particular application, the various disclosed embodiments are ideally suited for use with a Wearable Cardioverter Defibrillator (“WCD”), which is a WMD particularly adapted for cardiac care patients who are at high risk of a dangerous cardiac event but who may not be eligible for an implantable defibrillator. FIGS. 18 and 19, described below, illustrate example WCDs in which embodiments of this disclosure may be implemented.

FIG. 18 illustrates an illustrative WCD 1800 having a belt 1802 structured to wrap around a torso of a patient below a chest area. The illustrative WCD 1800 also includes two adjustable straps 1806 that extend over the patient's shoulders joining the back of the WCD 1800 to the front of the WCD 1800. The illustrative WCD 1800 may be worn by a patient of any gender, including a patient who prefers breast support. The illustrative WCD 1800 may, but need not, include breast support portions 1804 for the patient's comfort. The illustrative WCD 1800 shown in FIG. 18 may further include components, such as those illustrated in FIG. 19, operative to capture patient information and, in some embodiments, deliver medical therapy, such as a defibrillation shock.

FIG. 19 illustrates components of another illustrative WCD 1900 in which embodiments of this disclosure may be implemented or incorporated. The WCD 1900 may include a back portion 1908 that extends from the belt 1902 to the straps 1906. The illustrative WCD 1900 may also include a number of electrodes 1910 along the belt 1902 and the back portion 1908. The electrodes 1910 may be used to monitor physiological characteristics of the patient, such as electrocardiogram (ECG) of the patient, and/or to deliver medical treatment to the patient, such as a defibrillation shock. Wires 1912 are used to connect the various electrodes and other electrical components, such as an electronic module for defibrillation.

The illustrative WCD 1900 may also include a receptacle located on the belt 1902. The receptacle may be structured to accept the electronic module to receive inputs from and send instructions to various defibrillation components. The receptacle may also contain electrical contacts to electrically connect various electrical components to the supportive clothing device that may be used by the defibrillator, such as electrodes and a control module, for example.

Other embodiments include combinations and sub-combinations of features described or shown in the drawings herein, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

What is claimed is:

1. An accessory for a wearable medical device (WMD), wherein the WMD includes a garment to be worn by a patient, the garment including sensors for monitoring physiological characteristics of the patient, the garment being configured to be worn around the torso of the patient, the accessory comprising:
a belt portion that can be secured over the garment, the belt portion configured to house one or more of the sensors, the belt portion further comprising a belt portion cushion;
a back portion that includes cushioning material for added patient comfort;
an upper portion that extends from the belt portion and that includes sides that are attachable to the garment;
wherein the accessory can be adjusted and tightened around the garment 101 such that the ECG electrodes from losing contact with the skin.

2. The accessory recited in claim 1, wherein the sensors for monitoring physiological characteristics of the patient comprise ECG electrodes.

3. The accessory recited in claim 1, wherein the upper portion can be repositioned along the belt portion to allow a desired alignment with an upper body of the patient for coverage and/or support.

4. The accessory recited in claim 1, wherein positioning of the upper portion along the belt portion and/or sides of the garment can be achieved by one or more fastening mechanisms.

5. The accessory recited in claim 4, wherein the fastening mechanism comprise clips, clasps, zippers, lacing, hook-and-loop fasteners, hooks, or any combination of the foregoing.

6. The accessory recited in claim 1, wherein the upper portion of the accessory is securable to the portion with additional straps extending from the sides of the upper portion.

7. The accessory recited in claim 1, further comprising a back cushion extender configured to extend from the back portion cushion and fold underneath a belt of the garment, the back cushion extender configured to be folded between the patient's skin and an inner portion (skin facing layer) of the garment belt.

8. The accessory recited in claim 1, wherein the upper portion further comprises one or more interchangeable straps which may be interchanged based on the patient's preferences.

9. The accessory recited in claim 8, wherein the interchangeable straps comprise one or more of shoulder straps, cross-straps, halter-like straps, or any combination of the foregoing.

10. The accessory recited in claim 1, wherein the upper portion is configured as a tank top.

11. The accessory recited in claim 1, wherein the upper portion is configured as a vest.

12. The accessory recited in claim 1, wherein the upper portion is configured with a flap that opens to provide access to the patient's breast.

13. The accessory recited in claim 1, wherein the upper portion is configured to receive one or more health-related accessories.

14. The accessory recited in claim 13, wherein the health-related accessories comprises one or more of padding, a breast prosthesis, fluid drainage absorbent, compression pad material, tubing for a drainage port, or any combination of the foregoing.

15. The accessory recited in claim 1, wherein the upper portion further includes a detachable modesty panel configured to provide additional coverage of the patient's chest.

16. The accessory recited in claim 1, further comprising an ancillary wrap accessory attachable to the sides of the upper portion and configured to be wrapped around the patient's chest to provide additional support for the patient's breasts.

17. The accessory recited in claim 16, wherein one end of the ancillary wrap accessory is attachable to an attachment point in the shoulder area of the patient.

18. The accessory recited in claim 17, wherein the attachment point is on the garment

19. The accessory recited in claim 1, further comprising an ancillary wrap accessory attachable the belt portion of the accessory on one end of the ancillary wrap accessory and to an attachment point in the shoulder area of the patient on another end of the ancillary wrap accessory.

20. The accessory recited in claim 19, wherein the attachment point is on the garment.
